(19) United States
(12) Patent Application Publication
(10) Pub. No.: US 2007/0100139 A1
(43) Pub. Date: May 3, 2007

(54) METHODS FOR CHLORINATING SUCROSE-6-ESTER

(75) Inventor: John C. Fry, Horsham (GB)

Correspondence Address: SNELL & WILMER L.L.P. (Main), 400 EAST VAN BUREN, ONE ARIZONA CENTER, PHOENIX, AZ, 85004-2202, US

(73) Assignee: HEALTHY BRANDS, LLC, Deerfield Beach, FL (US)

(21) Appl. No.: 11/554,398

(22) Filed: Oct. 30, 2006

Related U.S. Application Data
(60) Provisional application No. 60/732,421, filed on Oct. 31, 2005.

(51) Int. Cl. C07H 13/02

(52) U.S. Cl. 536/119

(57) ABSTRACT

Methods for chlorinating sucrose-6-esters to produce 1′,4,6′-trichlorosucrose-6-esters include providing a reaction mixture in a temperature-controlled vessel at a temperature less than about 65° C., the reaction mixture comprising sucrose-6-ester, a tertiary amide, and a chloroformiminium chloride salt, which forms an O-alkylformiminium chloride adduct with the hydroxyl groups of the sucrose-6-ester. The methods for chlorinating the sucrose-6-ester further include subjecting the chloroformiminium chloride salt, tertiary amide, and sucrose-6-ester reaction mixture to an elevated temperature between about 75° C. and 100° C. for a period of time sufficient to produce a chlorinated product mixture of chlorinated sucrose-6-ester products consisting essentially of 1′,4,6′-trichlorogalacto-sucrose-6-ester.

### TECHNICAL FIELD

The invention relates to an improved process for the chlorination of sucrose-6-esters to produce selectively chlorinated products.

### BACKGROUND

The selective chlorination of less than all the hydroxyl groups of sucrose can be a major synthesis problem because the hydroxyl groups are of differing reactivity. The high potency sweetener sucralose, a compound whose formal name is 4-chloro-4-deoxy-α-D-galactopyranosyl-1,6-dichloro-1,6-dideoxy-β-D-fructofuranoside, is a partially chlorinated derivative of sucrose having chlorine substituted for the hydroxyl groups in the 6′, 4, and 1′ positions. While the selective chlorination of only the desired 6′, 4, and 1′ positions to produce sucralose began as a very complicated and challenging synthesis problem, many successful methods have been developed in the past several years to produce sucralose, through different synthetic routes or through different procedural steps, of differing yields and/or purities.

The initial process disclosed in the literature for the synthesis of sucralose involved the full selective protection of all the hydroxyl groups on the sucrose as follows:
(1) tritylation of sucrose at the 6, 1′, and 6′ primary hydroxyl groups with trityl chloride in pyridine;
(2) acetylation of the tri-tritylsucrose at the 5 secondary positions;
(3) removal of the trityl groups to give 2,3,4,3′,4′pentaacetylsucrose;
(4) migration of the acetyl group on the 4-position to the 6-position to produce 2,3,6,3′,4′-pentaacetyl-sucrose;
(5) chlorination of the free hydroxyls to produce sucralose pentaacetate; and
(6) deacetylation of the sucralose pentaacetate.
   The above-described process is disclosed, for example, by P. H. Fairclough, L. Hough, and A. C. Richardson, Carbohydr. Res., 40, 285 (1975); L. Hough, S. P. Phadnis, R. Khan, and M. R. Jenner, British Patents Nos. 1,543,167 and 1,543,168 (1979).

Considerable work has been carried out to determine the relative reactivities of the sucrose hydroxyl groups to chlorination. See, for instance, L. Hough, S. P. Phadnis, and E. Tarelli, Carbohydr. Res., 44, 35 (1975). The results indicate that the reactivity is 6 and 6′>4>1′>4′>others. Accordingly, a mild chlorination yields 6,6′-dichlorosucrose, a more vigorous chlorination gives 4,6,6′-trichloro sucrose (the 4-position is chlorinated with inversion of configuration, hence the product is 4,6,6′-trichloro-4,6,6′-trideoxygalactosucrose), and increasingly vigorous chlorinations give successively 4,6,1′,6′-tetrachloro-4,6,1′,6′-tetradeoxygalactosucrose and 4,6,1′,4′,6′-pentachloro-4,6,1′,4′,6′-pentadeoxygalactosucrose. Therefore, it is known that blocking the 6-position with a readily removable protecting group, such as a benzoate or an acetate ester group, followed by trichlorination and removal of the protecting group, will yield sucralose without the need for full protection of all the hydroxyl groups.

Prior methods of chlorinating a sucrose molecule with a blocked 6-position included combining a chlorinating reagent, either an acid chloride or a chloroformiminium chloride salt, with sucrose-6-ester in a tertiary amide reaction medium. Early patents, such as U.S. Pat. No. 4,380,476, issued to Mufti, et al., the entire disclosure of which is incorporated herein by reference for all purposes, disclose that sucrose-6-ester was added to a reaction medium including a chlorinating reagent. Mufti, et al disclosed that the temperature of the reaction medium was held below 50° C. prior to addition of the sucrose-6-ester, and after the addition of the sucrose-6-ester the temperature of the reaction medium was raised and held between 100° C. and 140° C.

Subsequently, Walkup et al. disclosed that a chlorinating reagent could be added to a tertiary amide reaction medium including the sucrose-6-ester. A full description of Walkup's methods are provided in U.S. Pat. No. 4,980,463, the entire disclosure being incorporated herein by reference for all purposes. In addition to inverting the order of addition of reagents to the reaction medium, Walkup disclosed that the temperature had to be carefully controlled during the addition of the chlorinating agent. Walkup also disclosed that the temperature should be increased in a step-wise manner to sequentially produce mono-, di-, and finally tri-chlorinated sucrose-6-ester.

Specifically, Walkup disclosed that the chlorination reaction should be carried out in two steps. The first step included holding the reaction medium at temperatures between 75° C. and 100° C., during which step Walkup disclosed that little or no tri-chlorination occurs, resulting in primarily mono- and some di-chlorinated sucrose-6-esters. Walkup further disclosed that “maintenance of the reaction mixture at this temperature for longer periods of time results in a higher degree of conversion of monochlorinated sucrose-6-esters to dichlorinated sucrose-6-esters with little or no trichlorination.” The second step in Walkup's chlorination method raised the temperature of the reaction mixture to temperatures in the range of 100° C. to 130° C. and held the temperature in that range to produce trichlorinated sucrose-6-ester. Walkup disclosed that the chlorination reaction generally occurred over a period ranging from five minutes to five hours, after which time the reaction was quenched by rapid cooling and the addition of an aqueous hydroxide solution to raise the pH. The quenching step had two functions. Firstly, to stop the chlorination reaction and so to limit the production of tetra- or penta-chlorinated sucrose-6-esters, and, secondly, to liberate the trichlorinated sucrose-6-ester from its complexed form of an O-alkylformiminium chloride adduct. FIGS. 4, 5, and 7 Of Walkup illustrate that continued chlorination beyond the desired reaction time results in a reduction in the recoverable yield of sucralose-6-ester, which reduction correlates with the illustrated increase in other tri-chlorinated and tetra-chlorinated sucrose-6-esters.

### SUMMARY

The present disclosure provides an improved process for the high-yield preparation of sucralose-6-esters by the controlled chlorination of sucrose-6-esters. The processes of the present disclosure include the steps of:
(a) providing a chlorination reaction mixture in a temperature-controlled vessel at a temperature less than about 65° C., the chlorination reaction mixture comprising sucrose-6-ester, a tertiary amide, and a chloroformiminium chloride salt, which forms an O-alkylformiminium chloride adduct with the hydroxyl groups of the sucrose-6-ester;
(b) subjecting the chlorination reaction mixture product of step (a) to an elevated temperature of at least about 75° C. and less than 100° C. for a period of time sufficient to produce a chlorinated product mixture of chlorinated sucrose-6-ester products consisting essentially of 1′,4,6′-trichlorogalacto-sucrose-6-ester O-alkylformiminium chloride adduct.

The chlorination reaction mixture of step (a) may be prepared through any suitable process. An exemplary process for preparing the chlorination reaction mixture includes adding a suitable amount of sucrose-6-ester to a reaction medium containing a chloroformiminium chloride salt in a tertiary amide medium. Additionally or alternatively, the chlorination reaction mixture of step (a) may be prepared by adding a suitable amount of acid chloride to a reaction medium comprising a tertiary amide and sucrose-6-ester to form a chloroformiminium chloride salt in solution. Other suitable processes may be used to prepare the chlorination reaction mixture of step (a).

### DETAILED DESCRIPTION

The present disclosure includes methods and systems for preparing sucralose-6-ester from sucrose-6-ester. The sucrose-6-ester starting material may be prepared through any suitable means to block the 6-position of the sucrose molecule. The methods and systems of the present disclosure provide a chlorination reaction mixture including a tertiary amide, a chloroformiminium chloride salt, and sucrose-6-ester in a temperature controlled reaction vessel to produce an O-alkylformiminium chloride adduct with the hydroxyl groups of the sucrose-6-ester. The temperature of the chlorination reaction mixture is then raised to a temperature sufficiently high to yield a mixture of chlorinated sucrose-6-esters consisting essentially of sucralose-6-ester while sufficiently low to substantially limit the production of tetrachlorinated sucrose-6-esters. For example, the temperature of the chlorination reaction mixture may be raised to between about 75° C. and 100° C.

To summarize the reactions that occur in the chlorination reaction of the present disclosure, the chlorination reaction mixture is prepared by combining sucrose-6-ester with a tertiary amide and a chloroformiminium chloride salt. The chloroformiminium chloride salt reacts with the unblocked hydroxyl groups of the sucrose-6-ester to form an O-alkylformiminium chloride adduct with one or more of the hydroxyl groups. HCl may be generated in this step of the reaction, which may form a complex with the tertiary amide of the reaction mixture. The O-alkylformiminium chloride adduct may be formed at one or more of the unblocked hydroxyl positions on the sucrose-6-ester. For example, three, four, five, six, or seven of the unblocked hydroxyl positions may react with the chloroformiminium chloride salt to form an O-alkylformiminium chloride adduct. When the O-alkylformiminium chloride adduct is heated, a rearrangement occurs at one or more of the carbon atoms of the sucrose-6-ester wherein the O-alkylformiminium chloride adduct is converted to a tertiary amide and a chlorinated sucrose-6-ester. The chlorinated sucrose-6-ester at this point in the reaction may also have one or more O-alkylformiminium chloride adducts associated with one or more of the hydroxyl groups that are not rearranged. As will be discussed in more detail below, the remaining chloride adducts are converted back to hydroxyl groups during the subsequent quenching step described below. Throughout this application, the chlorinated sucrose-6-ester, including the sucralose-6-ester, in the chlorination reaction mixture may be referred to herein as a simple chlorinated sucrose-6-ester with the adducts at the unchlorinated sites being understood but not specifically mentioned.

The position of the O-alkylformiminium chloride adduct on the sucrose-6-ester that is converted determines the position of the chlorination on the sucrose-6-ester. For example, the 6′ position is more reactive than the 1′ position and will be converted to form the 6′-monochlorinated-sucrose-6-ester at a different rate and under different conditions than would be necessary to produce a 1′-monochlorinated-sucrose-6-ester. The relative reactivities of the various hydroxyl groups are known and are described above. As described above, the predominant conversions proceed to chlorinate the 6′-position, the 4-position, the 1′-position, followed by the 4′-position and the other hydroxyl positions. The methods and systems of the present disclosure accomplish tri-chlorination of sucrose-6-ester at reaction temperatures between about 75° C. and about 100° C. to maximize the production of sucralose-6-ester while limiting the production of other tri-chlorinated sucrose-6-esters and tetra-chlorinated sucrose-6-esters.

As discussed briefly above, the chlorination reaction may occur in a chlorination reaction mixture prepared in any suitable manner. As used herein, the term “chlorination reaction mixture” refers to a mixture that includes at least a chloroformiminium chloride salt, sucrose-6-ester, and a tertiary amide while the term reaction medium refers to any mixture wherein a reaction may occur. A suitable preparation method results in a chlorination reaction mixture comprising a chloroformiminium chloride salt, sucrose-6-ester, and a tertiary amide. In one exemplary preparation method, the chloroformiminium chloride salt may be formed in situ by adding an acid chloride to a solution of sucrose-6-ester in a tertiary amide reaction medium. For example, phosgene may be added to a reaction medium of sucrose-6-ester and N,N-dimethylformamide (DMF). In another exemplary preparation method, an acid chloride and a tertiary amide may be combined to produce the chloroformiminium chloride salt in a tertiary amide reaction medium, to which the sucrose-6-ester may be added. In still another exemplary preparation method, a previously prepared chloroformiminium chloride salt may be added to a tertiary amide reaction medium, either before or after adding the sucrose-6-ester. Each of these exemplary preparation methods will be described in more detail below using specific acid chlorides, tertiary amides, chloroformiminium chloride salts, and sucrose-6-esters. While the preparation methods are discussed below using specific components, other components may be used. For example, phosgene as well as other suitable acid chlorides may be used. Moreover, while these three exemplary methods are described in the present application, other suitable methods of preparing a chlorination reaction mixture comprising a chloroformiminium chloride salt, sucrose-6-ester, and a tertiary amide may be used and are within the scope of the present disclosure.

The sucrose-6-ester may include sucrose-6-benzoate and sucrose-6-alkanoates, such as sucrose-6-acetate and the like. The purpose of the 6-ester group is simply to shield the hydroxyl on the 6-position of the sucrose molecule from the chlorination reaction. Accordingly, any ester group that is stable to the conditions of the chlorination reaction and which can be removed by hydrolysis under conditions that do not affect the remainder of the tri-chlorinated sucrose can be employed.

N,N-dimethylformamide (DMF) is one exemplary tertiary amide for use as a reaction medium in the methods of the present disclosure. Other tertiary amides that possess N-formyl groups, such as N-formylpiperidine, N-formylmorpholine, N,N-diethylformamide, and the like, can be employed in the process. Inert diluents, such as toluene, o-xylene, 1,1,2-trichloroethane, 1,2-diethoxyethane, diglyme (diethylene glycol dimethyl ether), and the like, can be employed at up to about 80 vol % or more of the liquid phase of the reaction medium, in addition to the tertiary amide. Useful cosolvents are those which are both chemically inert and which provide sufficient solvent power to enable the reaction to become essentially homogeneous during the chlorination reaction. Cosolvents with boiling points substantially below the chlorination reaction temperatures of the present disclosure may be employed.

Several acid chlorides, including phosgene, are known to form chloroformiminium chloride salts when reacted with tertiary amides and may be used as chlorine sources in the methods of the present disclosure. These acid chlorides include phosphorous oxychloride, phosphorous pentachloride, thionyl chloride, oxalyl chloride, methanesulfonyl chloride, and the like. As suggested above, the chloroformiminium chloride salt may be formed in situ, meaning in the presence of sucrose-6-ester, or it may be formed by reacting the acid chloride and tertiary amide prior to the addition of sucrose-6-ester to the reaction medium.

Additionally or alternatively, the chloroformiminium chloride salt may be provided in solid form or otherwise, having been previously prepared for use in the present methods or for other uses. Solid chloroformiminium chloride salts are commonplace and available for purchase from a number of sources. Suitable chloroformiminium chloride salts for the present methods include N,N-dimethylchloroformiminium chloride, also known as Arnold's Reagent, which is formed by the reaction of phosgene with DMF. Other suitable chloroformiminium chloride salts for the present methods include Vilsmeier-type salts formed from the reaction of an acid chloride with an N-formyl tertiary amide.

As indicated above, one method of preparing the chlorination reaction mixture of the present disclosure includes the formation of the chloroformiminium chloride salt in situ with the tertiary amide and the sucrose-6-ester. As used herein, the in situ formation of the chloroformiminium chloride salt refers to the addition of an acid chloride to a reaction medium including a tertiary amide and sucrose-6-ester, rather than the tertiary amide alone. The use of a tertiary amide as both a reaction solvent and a substrate for chloroformiminium chloride salt formation allows for the in situ formation of the chloroformiminium chloride salt.

A generalized description of the in situ chloroformiminium chloride salt formation method of preparing the chlorination reaction mixture of the present disclosure is set forth below, using phosgene as the acid chloride, DMF as the N-formyl tertiary amide, and sucrose-6-benzoate as the illustrative sucrose-6-ester. Other suitable acid chlorides, tertiary amides, and sucrose-6-esters may be used in suitable in situ formation methods.

Sucrose-6-benzoate is dissolved in two and one-half to five volumes of DMF and cooled to about 20° C. or lower. (Note that, as used herein, “volumes of solvent” is defined as liters of solvent per one kilogram of sucrose-6-benzoate, and all temperatures given are internal reaction temperatures.) For example, the sucrose-6-benzoate/DMF reaction medium may be cooled to between 10° C. and 20° C., to less than 10° C., or to less than 0° C. The sucrose-6-benzoate/DMF reaction medium may be stirred or otherwise agitated to induce mixing. A 50 to 75 wt. % solution of phosgene (7.5-11 molar equivalents relative to sucrose-6-benzoate) in toluene is then rapidly added with efficient agitation. Alternatively, pure phosgene may be added directly without toluene. Since sucrose-6-esters, such as sucrose-6-benzoate and sucrose-6-acetate, have seven free hydroxyl groups, at least seven molar equivalents of the acid chloride are employed to produce sufficient chloroformiminium chloride salt in the reaction medium to derivatize each free hydroxyl group on the sucrose-6-benzoate, even though only the three most reactive hydroxyl groups (positions 4,1′, and 6′) will ultimately undergo rearrangement to form the chlorinated sucrose-6-ester.

The phosgene addition is strongly exothermic due to the formation of N,N-dimethylchloroformiminium chloride and the reaction of this salt with the sucrose-6-benzoate hydroxyl groups to form the O-alkylformiminium chloride adduct discussed above. Accordingly, depending on the initial temperatures of the reagents (such as the cooling of the sucrose-6-benzoate/DMF to under 20° C. described above), more or less continued cooling may be required during the addition of the phosgene since attaining temperatures greater than about 60-70° C. during the addition can adversely affect the course of the reaction. Without being bound by theory, it is presently believed that maintaining the temperature below about 10-20° C. may result in improved yields. Again without being bound by theory, the improved yields are believed to result at least in part because of the slower formation of the chloroformiminium chloride, thereby allowing a more controlled and gentler formation of the O-alkylformiminium adduct. The lower temperature during the addition of the acid chloride may improve yields for other reasons as well. Easily stirred solids are formed in the reaction medium during the phosgene addition.

The reaction temperature then may be raised over a suitable period of time to a threshold temperature sufficient to effect substantial mono-chlorination of the sucrose-6-ester, as evidenced by the complete dissolution of all solids in the reaction vessel. Temperatures at which this occurs are found within the range of 50° C. to about 70° C., but typically from about 60° C. to about 65° C. The chlorination reaction mixture becomes homogeneous at this point and monochlorinated sucrose-6-benzoate derivatives are seen upon silica-gel TLC analysis (4.00:0.85:0.15, CHCl₃—CH₃OH—HOAC) of a worked-up reaction aliquot. The chlorination reaction mixture thus produced may be maintained at this temperature for at least one hour with little or no di- or higher chlorination occurring. In some aspects of the present disclosure, the internal temperature may be raised further substantially immediately upon attaining a homogeneous chlorination reaction mixture. Alternatively, the chlorination reaction mixture may be held in the range of about 50° C. to about 70° C. for some time beyond the formation of a homogeneous reaction mixture. Still alternatively, the chlorination reaction mixture may be raised directly to temperatures between about 75° C. and about 100° C. to effect the chlorination to form tri-chlorinated sucrose without pausing in the lower range of 50° C. to 70° C. While pausing to achieving homogeneity prior to heating above about 75° C. is not detrimental to the reaction or its outcome, it is not presently believed to be necessary.

As described briefly above, the chlorination reaction mixture of the present disclosure may also be prepared by adding an acid chloride to a tertiary amide, allowing the acid chloride and tertiary amide to react and form a chloroformiminium chloride salt in a tertiary amide reaction medium, and adding sucrose-6-ester to the chloroformiminium chloride salt and tertiary amide reaction medium. Any suitable combination of acid chlorides and tertiary amides may be reacted to form the chloroformiminium chloride salt. The combination of the acid chloride and the tertiary amide may produce chloroformiminium chloride salt solids in a tertiary amide reaction medium. The resulting solids may be further dissolved through agitation, heating, addition of tertiary amide, or some combination of dissolution steps prior to and/or during the addition of the sucrose-6-ester. Additionally or alternatively, chloroformiminium chloride salt from other sources may be added directly to a tertiary amide reaction medium. The chloroformiminium chloride salts may come from other providers or may come from other portions of the operator's facilities.

Accordingly, a reaction medium of chloroformiminium chloride salt and a tertiary amide may be prepared either by the addition of a previously prepared Vilsmeier-type salt to a tertiary amide reaction medium or by the addition of an acid chloride to a tertiary amide. In either scenario, sufficient acid chloride or Vilsmeier-type salt may be added to the tertiary amide reaction medium to derivative each of the seven free hydroxyl groups on the sucrose-6-ester. As described above, at least seven molar equivalents of acid chloride or Vilsmeier-type salt may be added to the tertiary amide reaction medium.

The addition of the acid chloride or the Vilsmeier-type salt to the tertiary amide may be exothermic. In implementations where the sucrose-6-ester is added to the reaction medium after the addition of the acid chloride and/or Vilsmeier-type salt, the internal reaction temperature is not critical during this stage of the reaction because the sucrose-6-ester is not in solution. Accordingly, the internal temperature of the reaction medium during the addition of the acid chloride or the addition of the Vilsmeier-type salt may be controlled, such as to a temperature range of less than 65° C., or may be uncontrolled. In some aspects of the present disclosure, the reaction medium internal temperature may be cooled to below about 65° C. prior to the addition of the sucrose-6-ester.

A sucrose-6-ester may be added to the tertiary amide and chloroformiminium chloride salt reaction medium, either alone or in a solvent, such as a tertiary amide. The addition of the sucrose-6-ester produces an O-alkylformiminium chloride adduct with the chloroformiminium chloride salt, which reaction is exothermic. Accordingly, the reaction medium may be cooled during the addition of the sucrose-6-ester. The internal reaction temperature may be controlled to below about 65° C. and the reaction medium may be stirred until substantially all the solids have dissolved and the solution becomes substantially homogenous.

An illustrative example of such a method where a sucrose-6-ester is added to a reaction medium including a tertiary amide and chloroformiminium chloride salt prepared by adding a Vilsmeier-type salt to a tertiary amide is described below. As previously described, alternative methods of preparing the reaction medium are within the scope of the present disclosure, including the use of other suitable tertiary amides, Vilsmeier-type salts, acid chlorides, and sucrose-6-esters.

A 500 ml jacketed glass reactor vessel was equipped with a reflux condenser topped with an Argon bubbler, an additional funnel, a thermometer, and a magnetic stirrer bar. Through the walls of the jacket was circulated a heat transfer fluid pumped in closed circuit through a thermostatic controller accurate to ±1° C. The reactor was charged with 90 ml dimethylformamide (DMF) and to this was added 22 g (171.9 mmol) of chloromethylene dimethyliminium chloride [(CH₃)₂N═CHCl]⁺Cl⁻, also known as a Vilsmeier-Haack-Arnold reagent or a Vilsmeier reagent (CAS number 3724-43-4).

Subsequently, 6 g (15.61 mmol) of sucrose-6-acetate was dissolved in 55 ml DMF. The temperature of the heat transfer fluid was set to 20° C. and the sucrose-6-acetate solution was added dropwise to the Vilsmeier reagent and DMF reaction medium with stirring. The dropping funnel was rinsed with an additional 5 ml DMF. During this process the internal reaction temperature rose to 30-40° C. The internal reaction temperature was then increased to 65° C. over about nine minutes and held at this point with continuous stirring until all solids had dissolved and the solution was substantially homogeneous, which took about ten minutes. The initial temperature of the heat transfer fluid is exemplary only and may be set to any suitable value sufficient to maintain the internal reaction temperature below about 65° C. until the solution is at least substantially homogeneous.

The chlorination reaction mixture of the present disclosure may also be prepared by addition of a Vilsmeier-type salt to a reaction medium containing a tertiary amide and a sucrose-6-ester. Such a preparation would proceed in a manner similar to the steps described above where the sucrose-6-ester is added to a reaction medium comprising a tertiary amide and a Vilsmeier-type salt. The reactions occurring in the reaction medium include the formation of the O-alkylformiminium chloride adduct between the Vilsmeier-type salt and the free hydroxyl groups of the sucrose-6-ester, which is the same reaction described in the previous illustrative example. Accordingly, the reaction will be similarly exothermic and will require similar amounts of the Vilsmeier-type salt to sufficiently derivative each of the free hydroxyl groups of the sucrose-6-ester. Similarly, the internal temperature of the reaction medium may be controlled to below about 65° C. during the addition of the Vilsmeier-type salt until the solution is at least substantially homogeneous.

The chlorination reaction mixture of the present disclosure may be prepared according to any one of the previously described manners, as well as other suitable manners. The chlorination reaction mixture thus prepared includes a tertiary amide, a sucrose-6-ester, and a chloroformiminium chloride salt, at least some of which may have formed an O-alkylformiminium chloride adduct with the free hydroxyl groups of the sucrose-6-ester. In some aspects of the present disclosure, the chlorination reaction mixture thus prepared may also include at least some mono-chlorinated sucrose-6-esters, such as 6′-monochloro-sucrose-6-ester.

The chlorination reaction mixture prepared according to the present disclosure may then be heated to between about 75° C. and 100° C. for a period of time sufficient to produce a chlorinated product mixture of chlorinated sucrose-6-esters consisting essentially of sucralose-6-ester, also known as 1′,4,6′-trichlorogalactosucrose-6-ester. As discussed briefly above, the product described here as sucralose-6-ester is actually a sucrose-6-ester chlorinated at the 1′,4, and 6′ sites and being an O-alkylformiminium chloride adduct at one or more of the remaining sites. The free sucralose-6-ester (without the adducts) is not formed until the subsequent quenching steps. The chlorination reaction mixture is held at this temperature for a period of time sufficient to maximize sucralose-6-ester production. During this time, sucralose-6-ester formation may be observed by silica gel TLC or HPLC of a suitably quenched sample from the reaction mixture. The temperature increase regimen for the tri-chlorination reaction may be conducted over a period of time ranging from about five minutes to about five hours prior to stabilizing between about 75° C. and 100° C., and more preferably between about 85° C. and 95° C.

In the above example where sucrose-6-acetate was added to the Vilsmeier reagent solution, the temperature of the heat transfer fluid was increased until an internal reaction temperature of 90° C. was reached (which took about twenty-six minutes) and this temperature was maintained (±1° C.) for two days. Early analytical results indicate that the yield of sucralose-6-acetate maximized at about one day. The time required to reach maximum sucralose-6-acetate yield may vary depending on the temperature of the chlorination reaction mixture and the specific Vilsmeier reagent being used. Without being bound by theory, it is presently believed that maximum sucralose-6-ester yield will be reached between about twelve hours and about fifty hours when the chlorination reaction is conducted between about 75° C. and 100° C., with increased temperatures reaching maximum yields in shorter time periods. For example, it is presently believed that the maximum sucralose-6-ester yield will be reached in about fifty hours when the chlorination reaction is carried out at about 85° C. and in about eighteen hours when the chlorination reaction is conducted at about 95° C.

As discussed above, increasingly vigorous chlorination accomplished through heating and stirring increases the rate of the chlorination reactions within the chlorination reaction mixture. For example, increasing the temperature of the reaction mixture yields some mono-chlorinated sucrose-6-esters, followed by conversion of substantially all of the sucrose-6-esters to at least mono-chlorinated sucrose-6-esters, with some being converted to di-chlorinated sucrose-6-esters. As discussed by Walkup et al., continual increases in temperature sequentially chlorinate the less reactive hydroxyl positions to form mono-, di-, tri-, tetra-, and other chlorinated sucrose-6-ester. Prior to the disclosure of the present application, it was believed that maintaining the temperature of the chlorination reaction mixture below 100° C. would not yield tri-chlorinated sucrose-6-ester. See Walkup et al., col. 6, lines 51-56. As described herein, tri-chlorinated sucrose-6-ester, and specifically sucralose-6-ester, can be produced at yields comparable to prior methods while maintaining the temperature of the chlorination reaction mixture between about 75° C. and 100° C.

Chlorination reaction mixtures within the scope of the present disclosure may include inert diluents and/or cosolvents such as described previously. Additionally or alternatively, chlorination reaction mixtures of the present disclosure may include one or more reagents other than the tertiary amides, chloroformiminium chloride salts, and sucrose-6-esters described above that may react with one or more of these reagents to affect the chlorination reaction. For example, one or more reagents may be added to the chlorination reaction mixture to alter and/or control the pH of the reaction mixture during the chlorination reaction. Other reagents may be added to improve or alter one or more reaction conditions.

One such reagent that may be added is acetic acid. In some implementations, the acetic acid may be added in concentrations between 0.25 vol. % and 5.0 vol. %. In one exemplary implementation, the acetic acid concentration may be about 1.0 vol. %. Without being bound by theory, it is presently believed that the addition of acetic acid to the chlorination reaction mixture may increase the yield of sucralose-6-ester. The acetic acid may be added to the chlorination reaction mixture at any step in the preparation of the reaction mixture. In some implementations, it may be preferred to have the acetic acid in the chlorination reaction mixture prior to having the chloroformiminium chloride salt and the sucrose-6-ester both in the reaction mixture. Additionally or alternatively, the acetic acid may be added to the reaction medium together, or substantially concurrently, with the last to be added of the sucrose-6-ester, the acid chloride, and/or the chloroformiminium chloride salt.

Conducting the chlorination reaction between about 75° C. and 100° C. is believed to lead to at least three significant improvements over prior methods of producing sucralose-6-ester where the chlorination reaction was raised to temperatures greater than 100° C. First, conducting the chlorination reaction in this lower temperature range reduces the occurrence of localized hot spots in the reaction vessel where the temperature may be high enough to produce tetra-chlorinated sucrose-6-esters. Second, conducting the chlorination reaction in this lower temperature range slows the chlorination reaction to a degree that accurate measurements of sucralose-6-ester yield may be made while the reaction is proceeding and the reaction may be quenched before the yield of sucralose-6-ester begins to decrease due to the production of tetra-chlorinated sucrose-6-esters and other byproducts. Third, it is presently believed that conducting the chlorination reaction at this lower temperature reduces the formation of tars, which may include uncharacterized carbohydrate breakdown products. These carbohydrate breakdown products reduce yield by wasting starting materials and complicating subsequent purification steps.

As described above, the chlorination reactions of the present disclosure are carried out in reaction vessels. At each stage in the reaction, the internal reaction temperature affects various aspects of the reaction, including the reaction rates and which reactions occur. The stirring of the chlorination reaction mixture and the application of a heat transfer fluid in the reaction vessel jacket may operate to maintain a fairly consistent temperature throughout the reaction mixture. However, the highly exothermic nature of the reactions is believed to produce localized hot spots, which may be temporary and/or transient within the reaction vessel. For example, the internal reaction temperature may be targeted for 90° C., but there may be localized hot spots with temperatures higher than 90° C. The extent to which the localized hot spots vary from the target temperature may depend on the reaction rates and the effectiveness of stirring the reaction mixture.

Without being bound by theory, it is presently believed that when the chlorination reaction is conducted at temperatures greater than 100° C., one or more of the localized hot spots may raise the temperature of a portion of the reaction mixture sufficiently to produce tetra-chlorinated sucrose-6-esters. It is believed that as the target temperature of the reaction mixture increases, the likelihood and frequency of hot spots with sufficiently high temperatures increases to increase the rate of tetra-chlorinated sucrose-6-ester production. By maintaining the target reaction mixture temperature between about 75° C. and 100° C., it is presently believed that the likelihood and frequency of hot spots having temperatures sufficiently hot to produce tetra-chlorinated sucrose-6-esters are reduced. Accordingly, it is believed that higher yields of tri-chlorinated sucrose-6-esters are possible. Moreover, the expected increased purity due to decreased production of tetra-chlorinated sucrose-6-ester may facilitate the remaining process steps to the production of sucralose.

Turning now to the second benefit provided by the methods of the present disclosure, conducting the chlorination reactions at internal temperatures between about 75° C. and about 100° C. enables greater control of the reactions. As briefly described above, the chlorination reaction to produce tri-chlorinated sucrose-6-ester requires quenching to stop the chlorination reaction, to limit tetra- and higher levels of chlorination, and to restore hydroxyl groups on the unchlorinated carbon atoms. Preferably, the chlorination reaction is quenched after the production and yield of sucralose-6-ester is maximized and before the yield is reduced through continued chlorination or other reactions. As the chlorination reaction proceeds, the quantity of sucralose-6-ester in solution may be measured through any suitable analytical technique. For example, one or more aliquots may be collected during the chlorination reaction and analyzed through one or more analytical techniques, such as silica-gel TLC analysis or HPLC analysis. Generally, the chlorination reaction progress is monitored by periodic sampling and analysis to determine when the reaction is at its peak yield. The analytical procedures alone can take from tens of minutes up to an hour or more to complete. Accordingly, a chlorination reaction where the peak yield of sucralose-6-ester is short-lived, such as shorter than 30 minutes or so, is inconveniently short due to the difficulty in identifying the point of maximum yield and stopping the reaction before the yield is reduced.

Without being bound by theory, it is presently believed that conducting the chlorination reaction under controlled temperatures between about 75° C. and 100° C. slows the chlorination reaction sufficiently to increase the amount of time during which the yield of sucralose-6-ester is at its peak. The lower temperature chlorination reaction decreases the production of tetra-chlorinated sucrose-6-ester and other tri-chlorinated sucrose-6-esters, which are believed to be the primary reactions that decrease the yield of sucralose-6-ester. Conducting the chlorination reaction in this lower internal temperature range is believed to prolong the peak yield time wherein the chlorination reaction is maintained in the vicinity of the maximum sucralose-6-ester yield. As used herein, peak yield time refers to the amount of time during which the sucralose-6-ester yield of the chlorination reaction is substantially close to the maximum yield obtainable before the yield begins to decrease, such as within 5% of the maximum yield. On an industrial scale, prolonging the peak yield time would give a better chance of stopping the reaction at an optimum time.

The time during the reaction at which the reaction is in the peak yield time as well as the duration of the peak yield time may vary depending on a number of reaction conditions. Sucralose-6-ester peak yield time durations of greater than 30 minutes are within the scope of this disclosure. In some implementations of the present methods, it is currently believed that the peak yield time duration may be longer than five hours and even as many long as ten or more hours. The lower chlorination reaction temperature prolongs the peak yield time duration to provide plenty of time to identify an optimum time to quench the reaction and maximize the yield. In comparison to prior methods that might only allow one or two samples within 5% of the maximum yield, the present methods allow multiple samples and analyses within the peak yield time. More preferably, the peak yield time will be between about 30 minutes and about 120 minutes. The temperature of the chlorination reaction within the range of the present disclosure may be selected based on a desired peak yield time, on a desired time to reach the peak yield time, or on a combination of the two factors.

Finally, as introduced above, the present methods are believed to reduce the production of tars during the chlorination reaction. Uncharacterized carbohydrate breakdown products are commonly described as tars due to their coloration. These tars present multiple problems to the chlorination reaction. First, the tars represent a waste of starting materials that could otherwise be converted to sucralose. Additionally, the tars complicate subsequent purification steps. Without being bound by theory, it is presently believed that the production of tars is tied to the temperature of the chlorination reaction. Accordingly, conducting the chlorination reaction at the lower temperatures of the present disclosure, which were previously understood to be too low to accomplish tri-chlorination, is believed to reduce the production of tars.

Chlorinating sucrose-6-ester at temperatures between about 75° C. and 100° C. may provide one or more of the advantages described above, though neither is required by the present disclosure. The methods of the present disclosure raise the temperature of a chlorination reaction mixture including a chloroformiminium chloride salt, a tertiary amide, and sucrose-6-ester to between about 75° C. and 100° C. to effect tri-chlorination of the sucrose-6-ester. The chlorination reaction of the present disclosure produces a chlorinated product mixture consisting essentially of sucralose-6-ester with the yield and purity of sucralose-6-ester at least comparable to those of prior methods.

At approximately the time the tri-chlorination yield is maximized, or at least within the peak yield time, the reaction mixture is “quenched” by being cooled to between about 0° C. and about 40° C. and rapidly treated with about 1.0 to 1.5 molar equivalents (relative to the acid chloride or chloroformiminium chloride salt) of cold aqueous alkali metal hydroxide, such as sodium or potassium hydroxide, or an aqueous slurry of an alkaline earth metal oxide or hydroxide, such as calcium oxide or calcium hydroxide. This neutralization or quenching is strongly exothermic. As excessively high temperatures will cause side reactions (e.g., anhydro derivative formation, de-esterification, etc.) resulting in a loss of sucralose-6-ester, temperatures are held below about 80° C. during this quenching operation. For optimum yield, the final pH of the reaction mixture is preferably within the range of about 8.5 to about 11, and preferably from about 9 to about 10.

The crude chlorination reaction product may also be quenched by adding the warm (75° C.-100° C.) DMF solution to about 1.0 to 1.5 molar equivalents (relative to the acid chloride or chloroformiminium chloride salt) of cold aqueous alkali such as sodium or potassium hydroxide, or a cold aqueous slurry of an alkaline-earth metal oxide or hydroxide, such as calcium oxide or calcium hydroxide, with vigorous agitation. As in the above-described neutralization method, control of pH and temperature is preferred in order to avoid diminished yields resulting from anhydro-sugar formation, de-esterification etc.

The chlorination reaction can also be quenched with concentrated aqueous or alcoholic ammonia using either mode of addition. This process, however, is less preferred because of the economic disadvantages inherent in the disposal of ammonia-containing wastes. Other suitable methods of quenching or stopping the chlorination reaction are within the scope of the present disclosure.

Once the chlorination reaction is sufficiently quenched, the sucralose-6-ester thus produced may be separated from the reaction mixture in a variety of suitable manners. Additionally, the sucralose-6-ester may be deacylated or deesterified through a number of suitable methods to produce the desired sucralose. Suitable separation and deacylation methods may be combined in either order. For example, the sucralose-6-ester may be separated from the remaining chlorinated sucrose-6-esters and then deesterified. Alternatively, the sucralose-6-ester may be deesterified to form sucralose and the sucralose then may be separated and purified. Exemplary separation, deacylation, and purification methods are described at least in Walkup et al. (U.S. Pat. No. 4,980,463), previously incorporated herein by reference; Navia et al. (U.S. Pat. No. 5,498,709), which is incorporated herein by reference in its entirety for all purposes; and U.S. patent application entitled CONVERSION OF SUCRALOSE-6-ESTER TO SUCRALOSE, filed on Sep. 27, 2005, naming John Charles Fry as inventor and assigned to Healthy Brands, LLC, which is incorporated herein by reference in its entirety for all purposes.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the description recites “a” or “a first” element or the equivalent thereof, such description should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, steps, functions, elements and/or properties may be claimed through amendment of the present claims or presentation of new claims in this or a related application. Such amended or new claims, whether they are directed to a different invention or directed to the same invention, whether different, broader, narrower or equal in scope to the original claims, are also regarded as included within the subject matter of the inventions of the present disclosure.

I claim:

1. A method of chlorinating sucrose-6-ester to produce 6′,4,1′-trichloro-sucrose-6-esters, the method comprising:
providing a chlorination reaction mixture comprising sucrose-6-ester, chloroformiminium chloride salt, and tertiary amide at a temperature below about 65° C., wherein at least a portion of the sucrose-6-ester and the chloroformiminium chloride salt forms an O-alkylformiminium chloride adduct;
heating the chlorination reaction mixture to between about 75° C. and about 100° C.;
maintaining the temperature of the chlorination reaction mixture between about 75° C. and about 100° C. to sustain a chlorination reaction for a sufficient time to produce a chlorinated sucrose product comprising predominantly 6′,4,1′-trichloro-sucrose-6-ester; and
quenching the chlorination reaction.

2. The method of claim 1, wherein the chlorination reaction mixture is prepared by adding a solid chloroformiminium chloride salt to a reaction medium comprising a tertiary amide and sucrose-6-ester while actively cooling the reaction medium to maintain the chlorination reaction mixture at a temperature below about 65° C. during the addition of the chloroformiminium chloride salt.

3. The method of claim 1, wherein the chlorination reaction mixture is prepared by adding sucrose-6-ester to a reaction medium comprising a chloroformiminium chloride salt and a tertiary amide while actively cooling the reaction medium to maintain the chlorination reaction mixture at temperatures below about 65° C. during the addition of sucrose-6-ester.

4. The method of claim 1, wherein the chlorination reaction mixture is prepared by adding an acid chloride to a reaction medium of sucrose-6-ester and a tertiary amide while actively cooling the reaction medium to maintain the chlorination reaction mixture at temperatures below about 65° C. during the addition of the acid chloride.

5. The method of claim 4, wherein the reaction medium is actively cooled to maintain the chlorination reaction mixture at temperatures below about 10° C. during the addition of the acid chloride.

6. The method of claim 1, wherein the temperature of the chlorination reaction mixture is maintained at temperatures between about 85° C. and about 95° C.

7. The method of claim 1, wherein the chlorination reaction time is between about 8 hours and about 70 hours.

8. The method of claim 1, wherein the temperature of the chlorination reaction mixture is maintained at about 85° C. and the chlorination reaction time is about 50 hours.

9. The method of claim 1, wherein the temperature of the chlorination reaction mixture is maintained at about 95° C. and the chlorination reaction time is about 12 hours.

10. The method of claim 1, wherein the chlorination reaction mixture additionally comprises acetic acid.

11. The method of claim 10, wherein the acetic acid is added to the chlorination reaction medium prior to forming the O-alkylformiminium chloride adduct between the chloroformiminium chloride salt and the sucrose-6-ester.

12. The method of claim 10, wherein the chlorination reaction mixture comprises between about 0.25 vol. % and about 5.0 vol. % acetic acid.

13. A method of chlorinating sucrose-6-ester to produce 6′,4,1′-trichloro-sucrose-6-esters, the method comprising:
providing a chlorination reaction mixture comprising sucrose-6-ester, chloroformiminium chloride salt, tertiary amide, and acetic acid at a temperature below about 65° C., wherein at least a portion of the sucrose-6-ester and the chloroformiminium chloride salt forms an O-alkylformiminium chloride adduct;
heating the chlorination reaction mixture to between about 85° C. and about 95° C.;
maintaining the temperature of the chlorination reaction mixture between about 85° C. and about 95° C. to sustain a chlorination reaction for a chlorination reaction time to produce a chlorinated sucrose product comprising predominantly 6′,4,1′-trichloro-sucrose-6-ester; and
quenching the chlorination reaction during a chlorination reaction peak yield time.

14. The method of claim 13, wherein the acetic acid is present in the chlorination reaction mixture at concentrations between about 0.5 vol. % and about 2.0 vol. %.

15. The method of claim 13, wherein the acetic acid is present in the chlorination reaction mixture at a concentration of about 1.0 vol. %.

16. The method of claim 13, wherein the peak yield time is between about 2 hours long and about 10 hours long.

17. The method of claim 13, wherein the chlorination reaction mixture is prepared by mixing sucrose-6-ester, acetic acid, and tertiary amide in a reaction medium followed by adding acid chloride to the reaction medium to form the chlorination reaction mixture, wherein the reaction medium is actively cooled to below about 65° C. during the addition of the acid chloride.

18. The method of claim 17, wherein the chlorination reaction mixture is cooled to below about 20° C. during the addition of the acid chloride.
